SEQUENCE LISTING (9 sequences)

SEQ ID NO: 1 - DNA, 1950 bp, synthetic construct
  source 1..1950: mol_type = other DNA
atgaccccaatcctgaccgtgctgatctgcctgggcctgtccctgggccctagaacacacgtgcaggccggccacctgcctaagcctaccctgtgggccgagcccggctccgttatcacacagggctcccccgtgaccctgagatgccagggcggccaggagacccaggagtacagactgtacagggagaagaagacagccctgtggatcacaaggattccccaggagctggtgaagaagggccagttccctatccctagcatcacatgggagcacgccggcagatacagatgttactacggctccgatacagccggcaggtccgagagctccgatcccctggagctggtggtgaccggcgcctacatcaagcccaccctgagcgcccagcctagccccgttgtgaatagcggcggcaatgtgatcctgcagtgtgacagccaggtggccttcgatggcttctccctgtgtaaggagggcgaggacgagcaccctcagtgcctgaatagccagccccacgccaggggctcctccagagctatcttcagcgtgggccccgtgtcccccagcaggagatggtggtacagatgctacgcctacgacagcaatagcccctacgagtggagcctgcccagcgacctgctggagctgctggtgctgggcgtgtccaagaagccctccctgagcgtgcagcccggccctatcgtggcccctgaggagacactgaccctgcagtgtggcagcgacgccggctacaataggttcgtgctgtacaaggacggcgagagggacttcctgcagctggccggcgcccagcctcaggctggactgtctcaggccaattttaccctgggccccgtgagcaggtcctacggcggacagtacagatgttatggcgcccacaatctgagctccgagtggtccgccccctccgatcccctcgatattctgatcgccggccagttttacgatagagtgagcctgagcgtgcaacccggccctaccgtggccagcggagagaacgtgacactgctgtgccagagccagggctggatgcagacctttctgctgaccaaggagggcgccgccgacgatccttggaggctgaggtccacataccagtcccagaagtaccaggccgagtttcctatgggccccgtgaccagcgcccacgctggaacatacagatgctatggcagccagtcctccaagccctacctgctgacccaccctagcgaccctctggagctggtcgtgtccggcccttccggcggaccttccagccctacaacaggccctacatccaccagcggccctgaggaccagcccctgacacctaccggcagcgacccccagtccggcctgggaagacacctgggcgtggtcatcggcatcctggtggccgtgatcctgctgctgctgctcctgctgctgctgttcctgatcctgaggcacagaagacagggcaagcactggacctccacacagaggaaggccgattttcagcaccctgccggcgccgtgggccctgagcctaccgacagaggcctgcagtggagaagctcccctgccgccgatgcccaggaggagaacctgtacgccgccgtgaagcacacacagcctgaggacggcgtggagatggacaccagatcccctcacgacgaggacccccaggccgtgacctacgccgaggtgaagcactccaggcccagaagagagatggccagcccccccagccctctgtccggagaatttctggatacaaaggacagacaggccgaggaggataggcagatggacaccgaggccgccgcctccgaggctcctcaagatgtgacatacgcccagctgcactccctgaccctgaggagagaggccaccgagccccccccatcccaggaaggaccctcccctgccgtgccttccatctacgccaccctggccatccac

SEQ ID NO: 2 - DNA, 879 bp, synthetic construct
  source 1..879: mol_type = other DNA
gatatcgtgatgacccaggccgccccctctgtgccagtcacccccggcgagagcgttagcatctcatgccgatcctccaagagcctgctccattcgaacggcaatacctacctgtattggttcctgcagcgtcccggccagagccctcagctgctgatttaccgcgtctcgaacctggcatctggggtccccgacaggttttctggctctgggtccggaactgcgttcaccctgcgcatctcccgggtggaggctgaagatgagggcgtgtactactgtatgcagcacttggagtacccattcaccttcggcgccggcaccaagctggagatcaagggcggctgcggcggatgtggtggttgcggcggctgtgcagggtgcggcggctgcggaggctgcgggggctgcggcggatgcgctggctgcggtggatgtgggggttgtggaggttgtggtgggtgcgccgggtgcggtggctgtggcggctgcgggggctgtgggggttgtgctggctgccaggtgcagctgaaacaatccggcgccgagctggctcgcccgggggcctccgtaaagttgtcctgtaaggcttctggctacaactttaaatcgtacggcatcagttgggtcaagcagcgcaccggacagggcctagaatggatcggtgagatttacccgggttcgggcaacacttattacaacgagaagctcaaaggtaaggccacccttacggcggacaagagctcgtccacagcctacatggagcttagaagtctcacctccgaggacagcgcggtgtatttttgtgcgcgcacatacggttacgactccttcccttggttcgcgtactggggccagggcactctggtgaccgtgagctct

SEQ ID NO: 3 - DNA, 888 bp, synthetic construct
  source 1..888: mol_type = other DNA
gacatcgtgatgacccaggccgctccctctgtaccggtgactccgggggagagcgtgtccatttcatgccgaagctcgaaatctctgctgcactccaacggcaacacgtacctgtactggttcctgcagcggcctggtcagagccctcagctgctcatctaccgcgttagcaacctggcctccggcgtgcccgacaggttttcaggctccggtagtggtactgcgttcacccttcgcatctcgcgcgtggaggccgaggacgtgggcgtctactactgtatgcaacacctggagtaccccttcacattcggaggagggaccaagctagagatcaagggcggctgcggtggttgtggcgggtgcggtgggtgcgcgggctgtggtggttgtggcggttgcggaggctgtggaggttgcgcagggtgtggcggctgcggtggctgcggagggtgcggcggctgtgcgggctgtggaggatgtggcggctgcgggggctgtggcggctgtgctgggtgccaggtccacttgcagcagagtggcaccgagctggtgaagcccggggcttccgttaagctgtcctgcgaggcatctggctacacgttcaccagctattggatgcattgggtcaagcagcgcccaggacagggtctcgaatggatcggcaacattaatccatcgaatggcggcaccaactacaacgagaaatttaagtccaaggccaccctgaccgtggacaagagctcctccaccgcctacatgcagctttcgtccttgacctccgaagattctgcggtgtactactgcgctcgtggcggcatctattacggcaactacgactattacgccatggattattggggccagggcacttcggtgacagtctcttct

SEQ ID NO: 4 - DNA, 54 bp, synthetic construct
  source 1..54: mol_type = other DNA
gagggaaggggcagcttattaacatgtggcgatgtggaagagaaccccggtccc

SEQ ID NO: 5 - DNA, 1488 bp, synthetic construct
  source 1..1488: mol_type = other DNA
atggccttaccagtgaccgccttgctcctgccgctggccttgctgctccacgccgccaggccggaggtgcagctggtggagagcggcggcggcctggtgcagcccggcggcagcctgaggctgagctgcgccgccagcggcttcgacctgggcttctacttctacgcctgctgggtgaggcaggcccccggcaagggcctggagtgggtgagctgcatctacaccgccggcagcggcagcacctactacgccagctgggccaagggcaggttcaccatcagcagggacaacagcaagaacaccctgtacctgcagatgaacagcctgagggccgaggacaccgccgtgtactactgcgccaggagcaccgccaacaccaggagcacctactacctgaacctgtggggccagggcaccctggtgaccgtgagcagcggcggcggcggcagcggcggcggcggcagcggcggcggcggcagcgacatccagatgacccagagccccagcagcctgagcgccagcgtgggcgacagggtgaccatcacctgccaggccagccagaggatcagcagctacctgagctggtaccagcagaagcccggcaaggtgcccaagctgctgatctacggcgccagcaccctggccagcggcgtgcccagcaggttcagcggcagcggcagcggcaccgacttcaccctgaccatcagcagcctgcagcccgaggacgtggccacctactactgccagagctacgcctacttcgacagcaacaactggcacgccttcggcggcggcaccaaggtggagatcaagaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctgataa

SEQ ID NO: 6 - DNA, 5194 bp, synthetic construct
  source 1..5194: mol_type = other DNA
acgcgtctggaacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattaattctgcagtcgagacctagaaaaacatggagcaatcacaagtagcaatacagcagctaccaatgctgattgtgcctggctagaagcacaagaggaggaggaggtgggttttccagtcacacctcaggtacctttaagaccaatgacttacaaggcagctgtagatcttagccactttttaaaagaaaagaggggactggaagggctaattcactcccaacgaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggccttgacattgctagcgtttaccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatcaacttgtttattgcagcttataatggttacaaataaagcaatagcatcacaaatttcacaaataaagcatttttttcactgcattctagttgtggtttgtccaaactcatcaatgtatcttatcatgtctggatcaactggataactcaagctaaccaaaatcatcccaaacttcccaccccataccctattaccactgccaattacctgtggtttcatttactctaaacctgtgattcctctgaattattttcattttaaagaaattgtatttgttaaatatgtactacaaacttagtagttggaagggctaattcactcccaaagaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccg

SEQ ID NO: 7 - DNA, 249 bp, synthetic construct
  source 1..249: mol_type = other DNA
ttcgtgcccgtgttcctgcccgccaaacctaccaccacccctgcccctagacctcccaccccagccccaacaatcgccagccagcctctgtctctgcggcccgaagcctgtagacctgctgccggcggagccgtgcacaccagaggcctggacttcgcctgcgacatctacatctgggcccctctggccggcacctgtggcgtgctgctgctgagcctggtgatcaccctgtactgcaaccaccggaac

SEQ ID NO: 8 - protein, 293 aa, synthetic construct
DIVMTQAAPSVPVTPGESVSISCRSSKSLLHSNGNTYLYWFLQRPGQSPQLLIYRVSNLASGVPDRFSGSGSGTAFTLRISRVEAEDEGVYYCMQHLEYPFTFGAGTKLEIKGGCGGCGGCGGCAGCGGCGGCGGCGGCAGCGGCGGCGGCGGCAGCGGCGGCGGCGGCAGCQVQLKQSGAELARPGASVKLSCKASGYNFKSYGISWVKQRTGQGLEWIGEIYPGSGNTYYNEKLKGKATLTADKSSSTAYMELRSLTSEDSAVYFCARTYGYDSFPWFAYWGQGTLVTVSS

SEQ ID NO: 9 - protein, 296 aa, synthetic construct
DIVMTQAAPSVPVTPGESVSISCRSSKSLLHSNGNTYLYWFLQRPGQSPQLLIYRVSNLASGVPDRFSGSGSGTAFTLRISRVEAEDVGVYYCMQHLEYPFTFGGGTKLEIKGGCGGCGGCGGCAGCGGCGGCGGCGGCAGCGGCGGCGGCGGCAGCGGCGGCGGCGGCAGCQVHLQQSGTELVKPGASVKLSCEASGYTFTSYWMHWVKQRPGQGLEWIGNINPSNGGTNYNEKFKSKATLTVDKSSSTAYMQLSSLTSEDSAVYYCARGGIYYGNYDYYAMDYWGQGTSVTVSS

(19) United States
(12) Patent Application Publication
(10) Pub. No.: US 2026/0183338 A1
(43) Pub. Date: Jul. 2, 2026

(54) RECOMBINANT VECTORS, LENTIVIRUSES, LUNG-EXEMPT MSLN CAR-T CELLS, AND ANTI-TUMOR AGENTS

(71) Applicant: TONGJI HOSPITAL, TONGJI MEDICAL COLLEGE OF HUAZHONG UNIVERSITY OF SCIENCE AND TECHNOLOGY, Wuhan (CN)

(72) Inventors: Yong FANG, Wuhan (CN); Ding MA, Wuhan (CN); Qinglei GAO, Wuhan (CN); Guang HU, Wuhan (CN); Xiaoyuan HUANG, Wuhan (CN); Minghua XIANG, Wuhan (CN); Huayi LI, Wuhan (CN); Wei MU, Wuhan (CN); Li ZHU, Wuhan (CN)

(73) Assignee: TONGJI HOSPITAL, TONGJI MEDICAL COLLEGE OF HUAZHONG UNIVERSITY OF SCIENCE AND TECHNOLOGY, Wuhan (CN)

(21) Appl. No.: 19/333,383

(22) Filed: Sep. 19, 2025

(30) Foreign Application Priority Data
Dec. 26, 2024 (CN) 202411934803.4

(51) Int. Cl. A61K 35/17; A61K 40/11; A61K 40/31; A61K 40/42; A61K 48/00; A61P 35/00; C07K 14/705; C07K 16/18; C07K 16/30; C12N 5/0783; C12N 15/86

(52) U.S. Cl. CPC A61K 35/17; A61K 40/11; A61K 40/31; A61K 40/4255; A61K 48/005; A61P 35/00; C07K 14/70503; C07K 16/18; C07K 16/30; C12N 5/0636; C12N 15/86; A61K 2239/13; A61K 2239/17; A61K 2239/29; C07K 2317/622; C12N 2510/00; C12N 2740/15043

(57) ABSTRACT

A recombinant vector, a lentivirus, a lung-exempt MSLN CAR-T cell, and an anti-tumor agent are provided. The recombinant vector includes a first nucleotide sequence, a second nucleotide sequence, and a third nucleotide sequence, the first nucleotide sequence encodes a mesothelin (MSLN)-binding domain, the second nucleotide sequence encodes a leukocyte immunoglobulin-like receptor (LIR-1), and the third nucleotide sequence specifically binds to a lung-highly-expressed molecule. The lentivirus is constructed using the recombinant vector. The lung-exempt MSLN CAR-T cell is constructed using the recombinant vector or the lentivirus. When the lung-exempt MSLN CAR-T cell enters lung tissue, the second nucleotide sequence binds to the lung-highly-expressed molecule through the third nucleotide sequence, inhibiting the killing function of CAR-T to avoid pulmonary toxicity; when the lung-exempt MSLN CAR-T cell infiltrates tumor tissue, the second nucleotide sequence is inactivated, activating CAR-T cells to kill tumor cells, offering the advantages of precise therapy and low side effects.

### CROSS-REFERENCE TO RELATED APPLICATIONS

This application claims priority of Chinese Application No. 202411934803.4, filed on Dec. 26, 2024, the entire contents of each of which are hereby incorporated by reference.

### SEQUENCE LISTING

The instant application contains a Sequence Listing which is submitted electronically in XML format and is hereby incorporated by reference in its entirety. The XML copy, created on Mar. 28, 2025, is named “2025 Mar. 28-Sequence List-67905-H005US00” and is 20,239 bytes in size.

### TECHNICAL FIELD

The present disclosure relates to the field of biogenetic engineering technology, and in particular, to a recombinant vector, a lentivirus, a lung-exempt MSLN CAR-T cell, and an anti-tumor agent.

### BACKGROUND

Mesothelin (MSLN) is expressed only on assignable tissues (limited to mesothelial cells of the pleura, peritoneum, and pericardium) and has very low nonspecific toxicity. Therefore, anti-tumor agents developed with MSLN as a target have low side effects and are currently of great interest.

For most cancers, MSLN is uniformly expressed on the cell surface (with lower expression in the cytoplasm). In particular, it shows high overexpression in mesothelioma, epithelial ovarian cancer, and pancreatic cancer. The aforementioned cancers are generally characterized by a high degree of malignancy and a high mortality rate. Moreover, due to the lack of typical symptoms in the early stage, patients are mostly diagnosed at a late stage, resulting in a poor prognosis. At present, surgery and chemotherapy are still the mainstay of treatment for these MSLN-targeted cancers, but due to the high recurrence rate and chemotherapy resistance, the overall efficacy is limited, and the survival rate of the patients is low. In recent years, immunotherapy, especially chimeric antigen receptor T-cell (CAR-T) therapy, has demonstrated significant efficacy in the treatment of a variety of solid tumors. Nevertheless, for the MSLN-targeted cancers, CAR-T therapy still faces a series of challenges, specifically, off-target toxicity is an important factor restricting the clinical application of CAR-T therapy.

Although MSLN is a highly specific and highly expressed target in MSLN-targeted cancers, it is highly expressed not only on cancer cells, but also in normal lung tissue at some level. Clinical trials have demonstrated that conventional MSLN-CAR-T therapy can trigger dose-dependent pulmonary toxicity reactions, leading to severe lung injury and even death in patients. This phenomenon significantly limits the clinical application of MSLN-CAR-T therapy. Therefore, there is an urgent need about how to reduce the pulmonary toxicity of MSLN-CAR-T therapy while maintaining its anti-cancer efficacy.

Therefore, it is desirable to provide a safe and effective lung-exempt MSLN CAR-T cell.

### SUMMARY

One or more embodiments of the present disclosure provide a recombinant vector, comprising a first nucleotide sequence, a second nucleotide sequence, and a third nucleotide sequence, wherein the first nucleotide sequence encodes a mesothelin (MSLN)-binding domain, the second nucleotide sequence encodes a leukocyte immunoglobulin-like receptor (LIR-1), and the third nucleotide sequence specifically binds to a lung-highly-expressed molecule.

In some embodiments, the lung-highly-expressed molecule is AQP3.

In some embodiments, the second nucleotide sequence is as shown in SEQ ID NO. 1.

In some embodiments, the third nucleotide sequence is as shown in SEQ ID NO. 2 or SEQ ID NO. 3.

In some embodiments, the first nucleotide sequence is as shown in SEQ ID NO. 5.

In some embodiments, a 5′ end of the second nucleotide sequence is sequentially linked to a HINGE sequence and the third nucleotide sequence.

In some embodiments, the HINGE sequence is as shown in SEQ ID NO. 7.

In some embodiments, a 3′ end of the second nucleotide sequence is sequentially linked to a T2A linker sequence, the first nucleotide sequence, and an activating sequence.

In some embodiments, the T2A linker sequence is as shown in SEQ ID NO. 4.

In some embodiments, the activating sequence is as shown in SEQ ID NO. 6.

One or more embodiments of the present disclosure provide a lentivirus. The lentivirus is constructed using the above recombinant vector.

One or more embodiments of the present disclosure provide a lung-exempt MSLN CAR-T cell. The lung-exempt MSLN CAR-T cell is constructed using the above recombinant vector.

One or more embodiments of the present disclosure provide a lung-exempt MSLN CAR-T cell. The lung-exempt MSLN CAR-T cell is constructed using the above recombinant vector.

One or more embodiments of the present disclosure provide an anti-tumor agent. The anti-tumor agent comprises the above lung-exempt MSLN CAR-T cell.

In some embodiments, the anti-tumor agent is selected from at least one of a drug, a pharmaceutical raw material, a vaccine, a targeted inhibitor, a gene therapy vector, and a biological material, and the agent further comprises a pharmaceutically acceptable excipient.

### BRIEF DESCRIPTION OF THE DRAWINGS

The present disclosure will be further illustrated by way of exemplary embodiments, which will be described in detail through the accompanying drawings, wherein:

FIG. 1 is a data comparison chart between single-cell sequencing data of normal lung tissue and single-cell sequencing data of ovarian cancer according to some embodiments of the present disclosure;

FIG. 2A is a schematic diagram illustrating co-localization of MSLN with AQP3, LAMP3, and SLC39A8 in ovarian cancer tissues according to some embodiments of the present disclosure;

FIG. 2B is a schematic diagram illustrating co-localization of MSLN with AQP3, LAMP3, and SLC39A8 in normal lung tissues according to some embodiments of the present disclosure;

FIG. 3 is a fluorescent image of cell lines according to some embodiments of the present disclosure;

FIG. 4 is a Western Blot image of cells collected after lentiviral transfection of a Hela cell line according to some embodiments of the present disclosure;

FIG. 5 is a fluorescence comparison image of the killing effect of CAR-T cell on the Hela-AQP3-GFP cell line and the Hela-mCherry cell line according to some embodiments of the present disclosure;

FIG. 6 is a comparison chart of the effects of CAR-T cell on the Hela-AQP3-GFP cell line and the Hela-mCherry cell line according to some embodiments of the present disclosure;

FIG. 7 is a schematic diagram illustrating flow cytometry analysis results of T cells according to some embodiments of the present disclosure;

FIG. 8 is a comparison graph of the different T cell contents in the experimental groups according to some embodiments of the present disclosure; and

FIG. 9 is a schematic diagram of the mechanism of a lung-exempt MSLN CAR-T cell according to some embodiments of the present disclosure.

### DETAILED DESCRIPTION

In order to provide a clearer understanding of the technical solutions of the embodiments described in the present disclosure, a brief introduction to the drawings required in the description of the embodiments is given below. It is evident that the drawings described below are merely some examples or embodiments of the present disclosure, and for those skilled in the art, the present disclosure may be applied to other similar situations without exercising creative labor.

As used in this description and the claims, unless the context clearly dictates otherwise, the words “a,” “an,” “one,” and/or “the” do not necessarily denote a singular or exclusive form, but may include plural forms as well. Generally, the terms “comprise” and “include” only indicate the inclusion of explicitly identified steps or elements, and such steps or elements do not form an exclusive list; a substance may also include other steps or elements.

One or more embodiments of the present disclosure provide a recombinant vector.

In some embodiments, the recombinant vector may include a first nucleotide sequence, a second nucleotide sequence, and a third nucleotide sequence. The recombinant vector refers to a deoxyribonucleic acid (DNA) molecule used to carry the target gene and transfer the target gene to the recipient cell in genetic engineering recombinant DNA technology.

In some embodiments, the first nucleotide sequence may be a nucleotide sequence encoding a mesothelin (MSLN) binding domain. Mesothelin is a cell surface glycoprotein encoded by the MSLN gene. It is rarely expressed in normal tissues but is overexpressed in various malignant tumors, including mesothelioma, ovarian cancer, pancreatic cancer, lung cancer, breast cancer, or the like.

In some embodiments, the first nucleotide sequence is as shown in SEQ ID NO. 5. The SEQ ID NO. 5 is ATGGCCTTACCAGTGACCGCCTTGCTCCTGCCGCTGGCCTTGCTGCTCCACGCCG CCAGGCCGGAGGTGCAGCTGGTGGAGAGCGGCGGCGGCCTGGTGCAGCCCGGC GGCAGCCTGAGGCTGAGCTGCGCCGCCAGCGGCTTCGACCTGGGCTTCTACTTC TACGCCTGCTGGGTGAGGCAGGCCCCCGGCAAGGGCCTGGAGTGGGTGAGCTG CATCTACACCGCCGGCAGCGGCAGCACCTACTACGCCAGCTGGGCCAAGGGCAG GTTCACCATCAGCAGGGACAACAGCAAGAACACCCTGTACCTGCAGATGAACAGC CTGAGGGCCGAGGACACCGCCGTGTACTACTGCGCCAGGAGCACCGCCAACACC AGGAGCACCTACTACCTGAACCTGTGGGGCCAGGGCACCCTGGTGACCGTGAGC AGCGGCGGCGGCGGCAGCGGCGGCGGCGGCAGCGGGGGGGGCAGCGACA TCCAGATGACCCAGAGCCCCAGCAGCCTGAGCGCCAGCGTGGGCGACAGGGTGA CCATCACCTGCCAGGCCAGCCAGAGGATCAGCAGCTACCTGAGCTGGTACCAGCA GAAGCCCGGCAAGGTGCCCAAGCTGCTGATCTACGGCGCCAGCACCCTGGCCAG CGGCGTGCCCAGCAGGTTCAGCGGCAGCGGCAGCGGCACCGACTTCACCCTGAC CATCAGCAGCCTGCAGCCCGAGGACGTGGCCACCTACTACTGCCAGAGCTACGC CTACTTCGACAGCAACAACTGGCACGCCTTCGGCGGCGGCACCAAGGTGGAGAT CAAGACCACGACGCCAGCGCCGCGACCACCAACACCGGCGCCCACCATCGCGTC GCAGCCCCTGTCCCTGCGCCCAGAGGCGTGCCGGCCAGCGGGGGGGGCGCAG TGCACACGAGGGGGCTGGACTTCGCCTGTGATATCTACATCTGGGCGCCCTTGGC CGGGACTTGTGGGGTCCTTCTCCTGTCACTGGTTATCACCCTTTACTGCAAACGGG GCAGAAAGAAACTCCTGTATATATTCAAACAACCATTTATGAGACCAGTACAAACTA CTCAAGAGGAAGATGGCTGTAGCTGCCGATTTCCAGAAGAAGAAGAAGGAGGATG TGAACTGAGAGTGAAGTTCAGCAGGAGCGCAGACGCCCCCGCGTACCAGCAGGG CCAGAACCAGCTCTATAACGAGCTCAATCTAGGACGAAGAGAGGAGTACGATGTTT TGGACAAGAGACGTGGCCGGGACCCTGAGATGGGGGGAAAGCCGAGAAGGAAGA ACCCTCAGGAAGGCCTGTACAATGAACTGCAGAAAGATAAGATGGCGGAGGCCTA CAGTGAGATTGGGATGAAAGGCGAGCGCCGGAGGGGCAAGGGGCACGATGGCC TTTACCAGGGTCTCAGTACAGCCACCAAGGACACCTACGACGCCCTTCACATGCA GGCCCTGCCCCCTCGCTGATAA.

In some embodiments, the second nucleotide sequence may be a nucleotide sequence encoding a leukocyte immunoglobulin-like receptor 1 (LIR-1). LIR-1 is an inhibitory receptor belonging to the leukocyte immunoglobulin-like receptor family (LIR family). LIR-1 is widely expressed on a variety of immune cells, including natural killer (NK) cells, dendritic cells, B cells, T cell, monocytes, and neutrophils. As an important immunosuppressive receptor, LIR-1 mainly binds to collagen to transmit inhibitory signals, thereby inhibiting the activation and function of immune cells, and widely participating in the regulation of immune cell function and the development of diseases.

The embodiments of the present disclosure introduce the nucleotide sequence encoding LIR-1 to achieve the transmission of signals from the AQP3-ScFv extracellular domain to the interior of cells, thereby inducing the generation of inhibitory signals to inhibit activity of the CAR-T cells.

In some embodiments, the second nucleotide sequence is as shown in SEQ ID NO. 1. The SEQ ID NO. 1 is

ATGACCCCAATCCTGACCGTGCTGATCTGCCTGGGCCTGTCCCTGGGCCCTAGAA
CACACGTGCAGGCCGGCCACCTGCCTAAGCCTACCCTGTGGGCCGAGCCCGGCT
CCGTTATCACACAGGGCTCCCCCGTGACCCTGAGATGCCAGGGCGGCCAGGAGA
CCCAGGAGTACAGACTGTACAGGGAGAAGAAGACAGCCCTGTGGATCACAAGGAT
TCCCCAGGAGCTGGTGAAGAAGGGCCAGTTCCCTATCCCTAGCATCACATGGGAG
CACGCCGGCAGATACAGATGTTACTACGGCTCCGATACAGCCGGCAGGTCCGAGA
GCTCCGATCCCCTGGAGCTGGTGGTGACCGGCGCCTACATCAAGCCCACCCTGA
GCGCCCAGCCTAGCCCCGTTGTGAATAGCGGCGGCAATGTGATCCTGCAGTGTGA
CAGCCAGGTGGCCTTCGATGGCTTCTCCCTGTGTAAGGAGGGCGAGGACGAGCA
CCCTCAGTGCCTGAATAGCCAGCCCCACGCCAGGGGCTCCTCCAGAGCTATCTTC
AGCGTGGGCCCCGTGTCCCCCAGCAGGAGATGGTGGTACAGATGCTACGCCTAC
GACAGCAATAGCCCCTACGAGTGGAGCCTGCCCAGCGACCTGCTGGAGCTGCTG
GTGCTGGGCGTGTCCAAGAAGCCCTCCCTGAGCGTGCAGCCCGGCCCTATCGTG
GCCCCTGAGGAGACACTGACCCTGCAGTGTGGCAGCGACGCCGGCTACAATAGG
TTCGTGCTGTACAAGGACGGCGAGAGGGACTTCCTGCAGCTGGCCGGCGCCCAG
CCTCAGGCTGGACTGTCTCAGGCCAATTTTACCCTGGGCCCCGTGAGCAGGTCCT
ACGGCGGACAGTACAGATGTTATGGCGCCCACAATCTGAGCTCCGAGTGGTCCGC
CCCCTCCGATCCCCTCGATATTCTGATCGCCGGCCAGTTTTACGATAGAGTGAGC
CTGAGCGTGCAACCCGGCCCTACCGTGGCCAGCGGAGAGAACGTGACACTGCTG
TGCCAGAGCCAGGGCTGGATGCAGACCTTTCTGCTGACCAAGGAGGGCGCCGCC
GACGATCCTTGGAGGCTGAGGTCCACATACCAGTCCCAGAAGTACCAGGCCGAGT
TTCCTATGGGCCCCGTGACCAGCGCCCACGCTGGAACATACAGATGCTATGGCAG
CCAGTCCTCCAAGCCCTACCTGCTGACCCACCCTAGCGACCCTCTGGAGCTGGTC
GTGTCCGGCCCTTCCGGCGGACCTTCCAGCCCTACAACAGGCCCTACATCCACCA
GCGGCCCTGAGGACCAGCCCCTGACACCTACCGGCAGCGACCCCCAGTCCGGCC
TGGGAAGACACCTGGGCGTGGTCATCGGCATCCTGGTGGCCGTGATCCTGCTGC
TGCTGCTCCTGCTGCTGCTGTTCCTGATCCTGAGGCACAGAAGACAGGGCAAGCA
CTGGACCTCCACACAGAGGAAGGCCGATTTTCAGCACCCTGCCGGCGCCGTGGG
CCCTGAGCCTACCGACAGAGGCCTGCAGTGGAGAAGCTCCCCTGCCGCCGATGC
CCAGGAGGAGAACCTGTACGCCGCCGTGAAGCACACACAGCCTGAGGACGGCGT
GGAGATGGACACCAGATCCCCTCACGACGAGGACCCCCAGGCCGTGACCTACGC
CGAGGTGAAGCACTCCAGGCCCAGAAGAGAGATGGCCAGCCCCCCCAGCCCTCT
GTCCGGAGAATTTCTGGATACAAAGGACAGACAGGCCGAGGAGGATAGGCAGATG
GACACCGAGGCCGCCGCCTCCGAGGCTCCTCAAGATGTGACATACGCCCAGCTG
CACTCCCTGACCCTGAGGAGAGAGGCCACCGAGCCCCCCCCATCCCAGGAAGGA
CCCTCCCCTGCCGTGCCTTCCATCTACGCCACCCTGGCCATCCAC.

In some embodiments, the 5′ end of the second nucleotide sequence is sequentially linked to a HINGE sequence and the third nucleotide sequence. The HINGE sequence is disposed between the second nucleotide sequence and the third nucleotide sequence for connecting the second nucleotide sequence and the third nucleotide sequence. The HINGE sequence refers to a flexible linker sequence in an antibody molecule that allows for better binding of the second nucleotide sequence and the third nucleotide sequence.

In some embodiments, the HINGE sequence is as shown in SEQ ID NO. 7. The SEQ ID NO. 7 is

TTCGTGCCCGTGTTCCTGCCCGCCAAACCTACCACCACCCCTGCCCCTA
GACCTCCCACCCCAGCCCCAACAATCGCCAGCCAGCCTCTGTCTCTGCG
GCCCGAAGCCTGTAGACCTGCTGCCGGCGGAGCCGTGCACACCAGAGGC
CTGGACTTCGCCTGCGACATCTACATCTGGGCCCCTCTGGCCGGCACCT
GTGGCGTGCTGCTGCTGAGCCTGGTGATCACCCTGTACTGCAACCACCG
GAAC.

In some embodiments, the 3′ end of the second nucleotide sequence is sequentially linked with a T2A linker sequence, the first nucleotide sequence, and an activating sequence. The T2A linker sequence is capable of linking two CAR molecules.

In some embodiments, the T2A linker sequence is as shown in SEQ ID NO. 4. The SEQ ID NO. 4 is

GAGGGAAGGGGCAGCTTATTAACATGTGGCGATGTGGAAGAGAACCCCG
GTCCC.

In some embodiments, the activating sequence is CD8a-CD28-CD3z.

The embodiments of the present disclosure introduce the activating sequence to achieve the transmission of stimulation signals from the MSLN-ScFv extracellular domain to the inside of the cells, thereby inducing the generation of activating signals to activate the cytotoxic effect of CAR-T cells.

In some embodiments, the activating sequence is shown as SEQ ID NO. 6. The SEQ ID NO. 6 is

ACGCGTCTGGAACAATCAACCTCTGGATTACAAAATTTGTGAAAGATTGACTGGTA
TTCTTAACTATGTTGCTCCTTTTACGCTATGTGGATACGCTGCTTTAATGCCTTTGT
ATCATGCTATTGCTTCCCGTATGGCTTTCATTTTCTCCTCCTTGTATAAATCCTGGTT
GCTGTCTCTTTATGAGGAGTTGTGGCCCGTTGTCAGGCAACGTGGCGTGGTGTGC
ACTGTGTTTGCTGACGCAACCCCCACTGGTTGGGGCATTGCCACCACCTGTCAGC
TCCTTTCCGGGACTTTCGCTTTCCCCCTCCCTATTGCCACGGCGGAACTCATCGCC
GCCTGCCTTGCCCGCTGCTGGACAGGGGCTCGGCTGTTGGGCACTGACAATTCC
GTGGTGTTGTCGGGGAAGCTGACGTCCTTTCCATGGCTGCTCGCCTGTGTTGCCA
CCTGGATTCTGCGCGGGACGTCCTTCTGCTACGTCCCTTCGGCCCTCAATCCAGC
GGACCTTCCTTCCCGCGGCCTGCTGCCGGCTCTGCGGCCTCTTCCGCGTCTTCGC
CTTCGCCCTCAGACGAGTCGGATCTCCCTTTGGGCCGCCTCCCCGCCTGGAATTA
ATTCTGCAGTCGAGACCTAGAAAAACATGGAGCAATCACAAGTAGCAATACAGCAG
CTACCAATGCTGATTGTGCCTGGCTAGAAGCACAAGAGGAGGAGGAGGTGGGTTT
TCCAGTCACACCTCAGGTACCTTTAAGACCAATGACTTACAAGGCAGCTGTAGATC
TTAGCCACTTTTTAAAAGAAAAGAGGGGACTGGAAGGGCTAATTCACTCCCAACGA
AGACAAGATATCCTTGATCTGTGGATCTACCACACACAAGGCTACTTCCCTGATTA
GCAGAACTACACACCAGGGCCAGGGGTCAGATATCCACTGACCTTTGGATGGTGC
TACAAGCTAGTACCAGTTGAGCCAGATAAGGTAGAAGAGGCCAATAAAGGAGAGA
ACACCAGCTTGTTACACCCTGTGAGCCTGCATGGGATGGATGACCCGGAGAGAGA
AGTGTTAGAGTGGAGGTTTGACAGCCGCCTAGCATTTCATCACGTGGCCCGAGAG
CTGCATCCGGAGTACTTCAAGAACTGCTGATATCGAGCTTGCTACAAGGGACTTTC
CGCTGGGGACTTTCCAGGGAGGCGTGGCCTGGGGGGACTGGGGAGTGGCGAG
CCCTCAGATCCTGCATATAAGCAGCTGCTTTTTGCCTGTACTGGGTCTCTCTGGTT
AGACCAGATCTGAGCCTGGGAGCTCTCTGGCTAACTAGGGAACCCACTGCTTAAG
CCTCAATAAAGCTTGCCTTGAGTGCTTCAAGTAGTGTGTGCCCGTCTGTTGTGTGA
CTCTGGTAACTAGAGATCCCTCAGACCCTTTTAGTCAGTGTGGAAAATCTCTAGCA
GTAGTAGTTCATGTCATCTTATTATTCAGTATTTATAACTTGCAAAGAAATGAATATC
AGAGAGTGAGAGGCCTTGACATTGCTAGCGTTTACCGTCGACCTCTAGCTAGAGC
TTGGCGTAATCATGGTCATAGCTGTTTCCTGTGTGAAATTGTTATCCGCTCACAATT
CCACACAACATACGAGCCGGAAGCATAAAGTGTAAAGCCTGGGGTGCCTAATGAG
TGAGCTAACTCACATTAATTGCGTTGCGCTCACTGCCCGCTTTCCAGTCGGGAAAC
CTGTCGTGCCAGCTGCATTAATGAATCGGCCAACGCGCGGGGAGAGGCGGTTTG
CGTATTGGGCGCTCTTCCGCTTCCTCGCTCACTGACTCGCTGCGCTCGGTCGTTC
GGCTGCGGCGAGCGGTATCAGCTCACTCAAAGGCGGTAATACGGTTATCCACAGA
ATCAGGGGATAACGCAGGAAAGAACATGTGAGCAAAAGGCCAGCAAAAGGCCAG
GAACCGTAAAAAGGCCGCGTTGCTGGCGTTTTTCCATAGGCTCCGCCCCCCTGAC
GAGCATCACAAAAATCGACGCTCAAGTCAGAGGTGGCGAAACCCGACAGGACTAT
AAAGATACCAGGCGTTTCCCCCTGGAAGCTCCCTCGTGCGCTCTCCTGTTCCGAC
CCTGCCGCTTACCGGATACCTGTCCGCCTTTCTCCCTTCGGGAAGCGTGGCGCTT
TCTCATAGCTCACGCTGTAGGTATCTCAGTTCGGTGTAGGTCGTTCGCTCCAAGCT
GGGCTGTGTGCACGAACCCCCCGTTCAGCCCGACCGCTGCGCCTTATCCGGTAA
CTATCGTCTTGAGTCCAACCCGGTAAGACACGACTTATCGCCACTGGCAGCAGCC
ACTGGTAACAGGATTAGCAGAGCGAGGTATGTAGGCGGTGCTACAGAGTTCTTGA
AGTGGTGGCCTAACTACGGCTACACTAGAAGAACAGTATTTGGTATCTGCGCTCTG
CTGAAGCCAGTTACCTTCGGAAAAAGAGTTGGTAGCTCTTGATCCGGCAAACAAAC
CACCGCTGGTAGCGGTGGTTTTTTTGTTTGCAAGCAGCAGATTACGCGCAGAAAAA
AAGGATCTCAAGAAGATCCTTTGATCTTTTCTACGGGGTCTGACGCTCAGTGGAAC
GAAAACTCACGTTAAGGGATTTTGGTCATGAGATTATCAAAAAGGATOTTCACCTAG
ATCCTTTTAAATTAAAAATGAAGTTTTAAATCAATCTAAAGTATATATGAGTAAACTT
GGTCTGACAGTTACCAATGCTTAATCAGTGAGGCACCTATCTCAGCGATCTGTCTA
TTTCGTTCATCCATAGTTGCCTGACTCCCCGTCGTGTAGATAACTACGATACGGGA
GGGCTTACCATCTGGCCCCAGTGCTGCAATGATACCGCGAGACCCACGCTCACCG
GCTCCAGATTTATCAGCAATAAACCAGCCAGCCGGAAGGGCCGAGCGCAGAAGTG
GTCCTGCAACTTTATCCGCCTCCATCCAGTCTATTAATTGTTGCCGGGAAGCTAGA
GTAAGTAGTTCGCCAGTTAATAGTTTGCGCAACGTTGTTGCCATTGCTACAGGCAT
CGTGGTGTCACGCTCGTCGTTTGGTATGGCTTCATTCAGCTCCGGTTCCCAACGAT
CAAGGCGAGTTACATGATCCCCCATGTTGTGCAAAAAAGCGGTTAGCTCCTTCGGT
CCTCCGATCGTTGTCAGAAGTAAGTTGGCCGCAGTGTTATCACTCATGGTTATGGC
AGCACTGCATAATTCTCTTACTGTCATGCCATCCGTAAGATGCTTTTCTGTGACTGG
TGAGTACTCAACCAAGTCATTCTGAGAATAGTGTATGCGGCGACCGAGTTGCTCTT
GCCCGGCGTCAATACGGGATAATACCGCGCCACATAGCAGAACTTTAAAAGTGCT
CATCATTGGAAAACGTTCTTCGGGGCGAAAACTCTCAAGGATCTTACCGCTGTTGA
GATCCAGTTCGATGTAACCCACTCGTGCACCCAACTGATCTTCAGCATCTTTTACTT
TCACCAGCGTTTCTGGGTGAGCAAAAACAGGAAGGCAAAATGCCGCAAAAAAGGG
AATAAGGGCGACACGGAAATGTTGAATACTCATACTCTTCCTTTTTCAATATTATTG
AAGCATTTATCAGGGTTATTGTCTCATGAGCGGATACATATTTGAATGTATTTAGAA
AAATAAACAAATAGGGGTTCCGCGCACATTTCCCCGAAAAGTGCCACCTGACGTC
GACGGATCGGGAGATCAACTTGTTTATTGCAGCTTATAATGGTTACAAATAAAGCA
ATAGCATCACAAATTTCACAAATAAAGCATTTTTTTCACTGCATTCTAGTTGTGGTTT
GTCCAAACTCATCAATGTATOTTATCATGTCTGGATCAACTGGATAACTCAAGCTAA
CCAAAATCATCCCAAACTTCCCACCCCATACCCTATTACCACTGCCAATTACCTGTG
GTTTCATTTACTCTAAACCTGTGATTCCTCTGAATTATTTTCATTTTAAAGAAATTGT
ATTTGTTAAATATGTACTACAAACTTAGTAGTTGGAAGGGCTAATTCACTCCCAAAG
AAGACAAGATATCCTTGATCTGTGGATCTACCACACACAAGGCTACTTCCCTGATT
AGCAGAACTACACACCAGGGCCAGGGGTCAGATATCCACTGACCTTTGGATGGTG
CTACAAGCTAGTACCAGTTGAGCCAGATAAGGTAGAAGAGGCCAATAAAGGAGAG
AACACCAGCTTGTTACACCCTGTGAGCCTGCATGGGATGGATGACCCGGAGAGAG
AAGTGTTAGAGTGGAGGTTTGACAGCCGCCTAGCATTTCATCACGTGGCCCGAGA
GCTGCATCCGGAGTACTTCAAGAACTGCTGATATCGAGCTTGCTACAAGGGACTTT
CCGCTGGGGACTTTCCAGGGAGGCGTGGCCTGGGGGGACTGGGGAGTGGCGA
GCCCTCAGATCCTGCATATAAGCAGCTGCTTTTTGCCTGTACTGGGTCTCTCTGGT
TAGACCAGATCTGAGCCTGGGAGCTCTCTGGCTAACTAGGGAACCCACTGOTTAA
GCCTCAATAAAGCTTGCCTTGAGTGCTTCAAGTAGTGTGTGCCCGTCTGTTGTGTG
ACTCTGGTAACTAGAGATCCCTCAGACCCTTTTAGTCAGTGTGGAAAATCTCTAGC
AGTGGCGCCCGAACAGGGACTTGAAAGCGAAAGGGAAACCAGAGGAGCTCTCTC
GACGCAGGACTCGGCTTGCTGAAGCGCGCACGGCAAGAGGCGAGGGGCGGCGA
CTGGTGAGTACGCCAAAAATTTTGACTAGCGGAGGCTAGAAGGAGAGAGATGGGT
GCGAGAGCGTCAGTATTAAGCGGGGGAGAATTAGATCGCGATGGGAAAAAATTCG
GTTAAGGCCAGGGGGAAAGAAAAAATATAAATTAAAACATATAGTATGGGCAAGCA
GGGAGCTAGAACGATTCGCAGTTAATCCTGGCCTGTTAGAAACATCAGAAGGCTG
TAGACAAATACTGGGACAGCTACAACCATCCCTTCAGACAGGATCAGAAGAACTTA
GATCATTATATAATACAGTAGCAACCCTCTATTGTGTGCATCAAAGGATAGAGATAA
AAGACACCAAGGAAGCTTTAGACAAGATAGAGGAAGAGCAAAACAAAAGTAAGAC
CACCGCACAGCAAGCGGCCG.

In some embodiments, based on the standard ligation direction or the nucleotide sequence (from the 5′ end to the 3′ end), the ligation structure of the recombinant vector may be the third nucleotide sequence, the HINGE sequence, the second nucleotide sequence, the T2A linker sequence, the first nucleotide sequence, and the activating sequence.

In some embodiments, the third nucleotide sequence is a nucleotide sequence for specific binding to a lung-highly-expressed molecule.

In some embodiments, the lung-highly-expressed molecule refers to an aquaporin-3 (AQP3). The AQP3 refers to a transmembrane protein belonging to the aquaporin family, which is highly expressed in lung tissues and poorly expressed in most cancerous tissues, including ovarian cancer.

In some embodiments, a single-chain antibody fragment (scFv) sequence encoded by the third nucleotide sequence may specifically bind to the lung-highly-expressed molecule.

It is understandable that the antibody-binding domain in the extracellular domain is typically an antibody-derived scFV, which is responsible for antigen recognition. The scFV consists of a variable heavy chain region (VH) and a variable light chain region (VL) of an antibody, connected by a peptide linker, and then linked to a transmembrane domain via a Hinge region.

In some embodiments, the third nucleotide sequence is shown as SEQ ID NO. 2 or SEQ ID NO. 3. The SEQ ID NO. 2 is GATATCGTGATGACCCAGGCCGCCCCCTCTGTGCCAGTCACCCCCGGCGAGAGC GTTAGCATCTCATGCCGATCCTCCAAGAGCCTGCTCCATTCGAACGGCAATACCTA CCTGTATTGGTTCCTGCAGCGTCCCGGCCAGAGCCCTCAGCTGCTGATTTACCGC GTCTCGAACCTGGCATCTGGGGTCCCCGACAGGTTTTCTGGCTCTGGGTCCGGAA CTGCGTTCACCCTGCGCATCTCCCGGGGGAGGCTGAAGATGAGGGCGTGTACTA CTGTATGCAGCACTTGGAGTACCCATTCACCTTCGGCGCCGGCACCAAGCTGGAG ATCAAGGGCGGCTGCGGCGGATGTGGTGGTTGCGGCGGCTGTGCAGGGTGCGG CGGCTGCGGAGGCTGCGGGGGCTGCGGCGGATGCGCTGGCTGCGGTGGATGTG GGGGTTGTGGAGGTTGTGGTGGGTGCGCCGGGTGCGGTGGCTGTGGCGGCTGC GGGGGCTGTGGGGGTTGTGCTGGCTGCCAGGTGCAGCTGAAACAATCCGGCGCC GAGCTGGCTCGCCCGGGGGCCTCCGTAAAGTTGTCCTGTAAGGCTTCTGGCTACA ACTTTAAATCGTACGGCATCAGTTGGGTCAAGCAGCGCACCGGACAGGGCCTAGA ATGGATCGGTGAGATTTACCCGGGTTCGGGCAACACTTATTACAACGAGAAGCTCA AAGGTAAGGCCACCCTTACGGCGGACAAGAGCTCGTCCACAGCCTACATGGAGCT TAGAAGTCTCACCTCCGAGGACAGCGCGGTGTATTTTTGTGCGCGCACATACGGT TACGACTCCTTCCCTTGGTTCGCGTACTGGGGCCAGGGCACTCTGGTGACCGTGA GCTCT. The SEQ ID NO. 3 is

GACATCGTGATGACCCAGGCCGCTCCCTCTGTACCGGTGACTCCGGGGGA
GAGCGTGTCCATTTCATGCCGAAGCTCGAAATCTCTGCTGCACTCCAACG
GCAACACGTACCTGTACTGGTTCCTGCAGCGGCCTGGTCAGAGCCCTCAG
CTGCTCATCTACCGCGTTAGCAACCTGGCCTCCGGCGTGCCCGACAGGTT
TTCAGGCTCCGGTAGTGGTACTGCGTTCACCCTTCGCATCTCGCGCGTGG
AGGCCGAGGACGTGGGCGTCTACTACTGTATGCAACACCTGGAGTACCCC
TTCACATTCGGAGGAGGGACCAAGCTAGAGATCAAGGGCGGCTGCGGTGG
TTGTGGGGGGTGCGGTGGGTGCGCGGGCTGTGGTGGTTGTGGCGGTTGCG
GAGGCTGTGGAGGTTGCGCAGGGTGTGGCGGCTGCGGTGGCTGCGGAGGG
TGCGGCGGCTGTGCGGGCTGTGGAGGATGTGGCGGCTGCGGGGGCTGTGG
CGGCTGTGCTGGGTGCCAGGTCCACTTGCAGCAGAGTGGCACCGAGCTGG
TGAAGCCCGGGGCTTCCGTTAAGCTGTCCTGCGAGGCATCTGGCTACACG
TTCACCAGCTATTGGATGCATTGGGTCAAGCAGCGCCCAGGACAGGGTCT
CGAATGGATCGGCAACATTAATCCATCGAATGGCGGCACCAACTACAACG
AGAAATTTAAGTCCAAGGCCACCCTGACCGTGGACAAGAGCTCCTCCACC
GCCTACATGCAGCTTTCGTCCTTGACCTCCGAAGATTCTGCGGTGTACTA
CTGCGCTCGTGGCGGCATCTATTACGGCAACTACGACTATTACGCCATGG
ATTATTGGGGCCAGGGCACTTCGGTGACAGTCTCTTCT.

The SEQ ID NO. 2 and the SEQ ID NO. 3 exhibit high affinity to the AQP3 peptide, and their antigen-antibody binding ability demonstrates significant superiority.

One or more embodiments of the present disclosure provide a lentivirus. In some embodiments, the lentivirus may be constructed using the recombinant vector as described in the embodiments of this disclosure.

One or more embodiments of the present disclosure provide a lung-exempt MSLN CAR-T cell. In some embodiments, the lung-exempt MSLN CAR-T cell may be constructed using the recombinant vector as described in the embodiments of the present disclosure. In some embodiments, the lung-exempt MSLN CAR-T cell may be constructed using the lentivirus as described in the embodiments of the present disclosure.

In the embodiment of the present disclosure, the lung-exempt MSLN CAR-T cell refers to a novel dual-targeting CAR-T cell, which is constructed using the recombinant vector or the lentivirus as described previously. The lung-exempt MSLN CAR-T cell not only includes a nucleotide sequence encoding the MSLN binding domain, but also includes a nucleotide sequence encoding the LIR-1 and a nucleotide sequence encoding the nucleotide sequence that specifically binds to a lung-highly-expressed molecule.

The introduction of the nucleotide sequence encoding the LIR-1 provides a new idea for optimizing the function of CAR-T cells. The nucleotide sequence encoding the LIR-1 can inhibit the activation of T cells by binding to specific molecules, thereby selectively reducing the activity of CAR-T cells and realizing precision therapy.

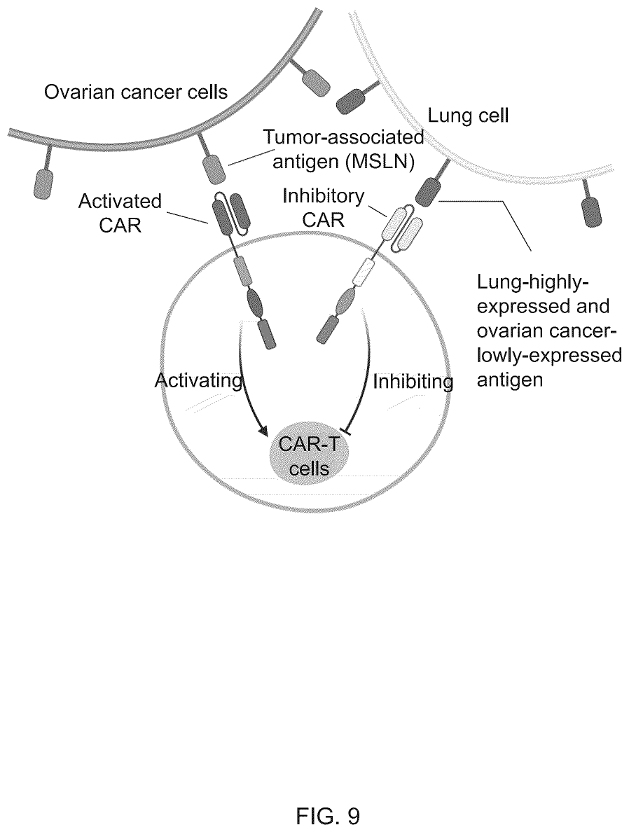

FIG. 9 is a schematic diagram of the mechanism of the lung-exempt MSLN CAR-T cell according to some embodiments of the present disclosure. As shown in FIG. 9, the lung-exempt MSLN CAR-T cell introduces the nucleotide sequence encoding the LIR-1 and the nucleotide sequence encoding the LIR-1 links to the lung-highly-expressed molecule. When the lung-exempt MSLN CAR-T cell enters lung tissue, the nucleotide sequence encoding the LIR-1 inhibits the killing function of the CAR-T cells by binding to the lung-highly-expressed molecule, thereby avoiding pulmonary toxicity. When the lung-exempt MSLN CAR-T cell infiltrates the cancerous tissue, the nucleotide sequence encoding the LIR-1 is inactivated, and the CAR-T cells are partially activated to function normally and effectively kill cancer cells.

In the embodiments of the present disclosure, the lung-exempt MSLN CAR-T cell can effectively reduce the off-target toxicity of CAR-T cells, providing a safer therapeutic strategy for the clinical application of the MSLN CAR-T cell.

In some embodiments, the lung-exempt MSLN CAR-T cell is used to prepare an anti-tumor agent, and the tumor is targeted at the MSLN.

One or more embodiments of the present disclosure provide the anti-tumor agent. In some embodiments, the anti-tumor agent comprises the lung-exempt MSLN CAR-T cell as described in the embodiments of the present disclosure, i.e., a cell constructed by the recombinant vector or the lentivirus.

In some embodiments, the anti-tumor agent targets the tumors that use the MSLN as a target. For example, the tumors include, but are not limited to, mesothelioma, ovarian cancer, pancreatic cancer, liver cancer, esophageal cancer, gastric cancer, peritoneal cancer, breast cancer, or the like.

In some embodiments, the anti-tumor agent may be selected from a drug, a pharmaceutical raw material, a vaccine, a targeted inhibitor, a gene therapy vector, or a biomaterial. In some embodiments, the anti-tumor agent may further include a pharmaceutically acceptable excipient.

The embodiments of the present disclosure have at least the following beneficial effects:
(1) The lung-exempt MSLN CAR-T cell introduces the nucleotide sequence encoding the LIR-1 and the nucleotide sequence encoding the LIR-1 links the lung-highly-expressed molecule. When the lung-exempt MSLN CAR-T cell enters the lung tissue, the nucleotide sequence encoding the LIR-1 inhibits the killing function of the CAR-T cells by binding to the lung-highly-expressed molecule, thereby avoiding pulmonary toxicity. When the lung-exempt MSLN CAR-T cell infiltrates the cancerous tissue, the nucleotide sequence encoding the LIR-1 is inactivated, and the CAR-T cells are partially activated to function normally and effectively kill the cancer cells;
(2) The lung-exempt MSLN CAR-T cell can realize precision therapy, effectively reduce the off-target toxicity of CAR-T cells, reduce side effects, and provide a safer therapeutic strategy for the clinical application of the MSLN CAR-T cell;
(3) The AQP3 is selected as the lung-highly-expressed molecule for the lung-exempt MSLN CAR-T cell. The AQP3 exhibits high-expression in the lung tissues and low-expression in the vast majority of the cancer tissues such as ovarian cancer, making it suitable for use as a target for a lung-exempt CAR-T cell, further enhancing the specificity and safety of this therapeutic strategy.

The lung-exempt MSLN CAR-T cell provided herein is described below by way of Example 1-Example 3.

### Example 1: Single Cell Data Screening

The rapid development of single-cell sequencing technology has provided an important tool for the discovery of differentially expressed genes between tumors and normal tissues. By analyzing transcriptome data from different tissues, molecules that are highly expressed in specific tissues but lowly expressed in tumors can be screened, thus providing a basis for CAR-T target optimization.

In this example, single-cell sequencing data from normal lung tissue and single-cell sequencing data from ovarian cancer were used for analysis to screen for genes that are highly expressed in the lung tissues and lowly expressed in the ovarian cancer tissues.

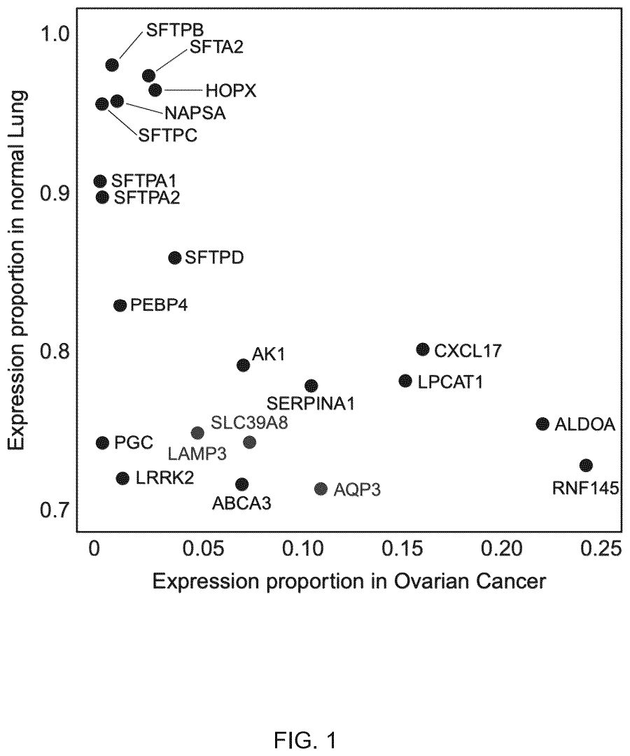

FIG. 1 is a data comparison chart between the single-cell sequencing data of normal lung tissue and the single-cell sequencing data of ovarian cancer according to some embodiments of the present disclosure.

As shown in FIG. 1, 21 candidate genes may be obtained through comparison screening of single-cell sequencing data. The screened candidate genes need to meet two requirements. On the one hand, the screened candidate genes are inhibitory antigens that are lowly expressed in ovarian cancer and highly expressed in lung tissues. On the other hand, the screened candidate genes are membrane-expressed proteins so that they can be recognized by CAR-T cells. Specifically, the single-cell sequencing datasets of ovarian cancer and normal lung tissues (Synapse ID for ovarian cancer single-cell sequencing data: syn25569736; Synapse ID for lung single-cell sequencing data: syn21041850) are downloaded respectively, bioinformatics analysis is performed the single-cell sequencing datasets using R studio to screened out 19,918 genes transcribed in less than 30% of ovarian cancer cells and 329 genes transcribed in more than 70% of normal lung cells, and 21 candidate genes are obtained by taking the intersection of these two gene sets. Subsequently, the Human Protein Atlas website is used to query the coding type and the cell expression localization information of each candidate gene and the protein expression level of the candidate gene in ovarian cancer and lung tissues. Since the CAR-T cells only recognize membrane-expressed antigens, the intracellularly expressed and secreted proteins and the known highly expressed proteins in ovarian cancer are excluded, and three genes are screened out, namely SLC39A8, LAMP3, and the AQP3.

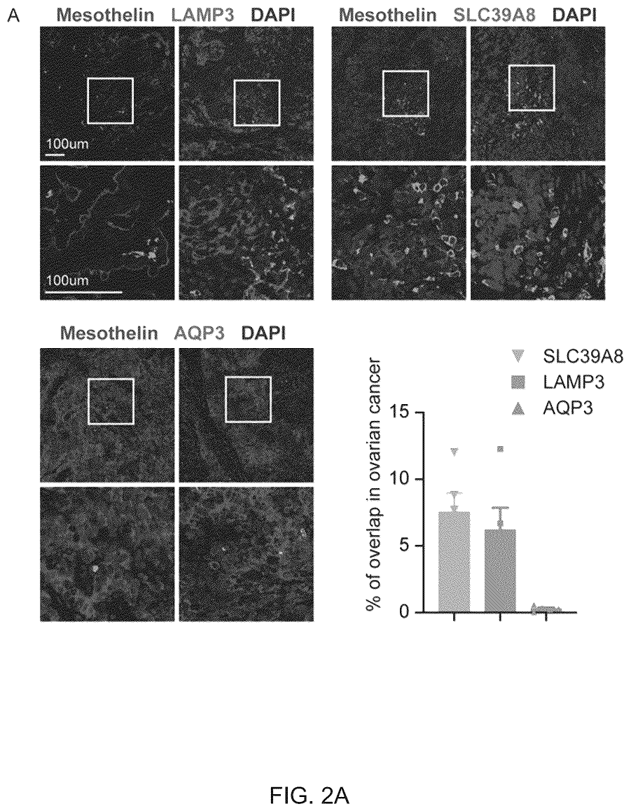

The expression of the aforementioned three genes in normal lung tissue and ovarian cancer may be verified by immunohistochemical experimental techniques. FIG. 2A is a schematic diagram illustrating co-localization of MSLN with AQP3, LAMP3, and SLC39A8 in ovarian cancer tissues according to some embodiments of the present disclosure. FIG. 2B is a schematic diagram illustrating co-localization of MSLN with AQP3, LAMP3, and SLC39A8 in normal lung tissues according to some embodiments of the present disclosure. According to FIGS. 2A-2B, it can be seen that the LAMP3 and the SLC39A8 are expressed at significantly higher levels than the AQP3 in ovarian cancer, so the LAMP3 and the SLC39A8 are excluded and the most suitable candidate gene is identified as the AQP3 (Aquaporin-3, Ensembl: ENSG00000165272).

### Example 2: Lentivirus Synthesis

In this example, a monoclonal antibody that specifically binds to the AQP3 extracellular domain and the MSLN binding domain were employed to construct plasmids AQP3-C scFV-LIR1-T2A-MSLN scFv-CD8a-CD28-CD3z and AQP3-J SCFV-LIR1-T2A-MSLN scFv-CD8a-CD28-CD3z for packaging the lentivirus, which were then used to infect human primary T cells.

The above sequences were synthesized into plasmids using a vector pXL0091, which was from Nanjing Reindeer Biotech Co., Ltd., and plasmid synthesis was done by Gemma Gene Co., Ltd. Lentiviral packaging was done by Nanjing GenScript Biotech Co., Ltd.

The specific steps for the infection of the human primary T cells using the obtained lentivirus were as follows:
(1) Before transfection, the density of T cells was adjusted to 10⁶ cells/mL, and 2 mL of fresh T cell complete medium was added to a six-well plate. 200 UL of viral stock solution and polybrene at a final concentration of 5 μg/mL were added to the six-well plate, and the plate was gently blown to mix well, and then cultured in an incubator at 37° C. with a CO₂ concentration of 5% for 24 hours.
(2) After completing the culture, the infection solution was replaced with fresh complete medium and the culture was continued in the incubator for 24 hours to 48 hours. After the infection is completed, in vitro killing experiments may be performed.

In the example, the sequences of the monoclonal antibodies that specifically binds to the AQP3 extracellular domain are shown in SEQ ID NO. 2 and SEQ ID NO. 3, respectively, and the amino acid sequences encoded by SEQ ID NO. 2 and SEQ ID NO. 3 are shown in SEQ ID NO. 8 and SEQ ID NO. 9, respectively. The SEQ ID NO. 8 is DIVMTQAAPSVPVTPGESVSISCRSSKSLLHSNGNTYLYWFLQRPGQSPQLLIYRVSNL ASGVPDRFSGSGSGTAFTLRISRVEAEDEGVYYCMQHLEYPFTFGAGTKLEIKGGCG GCGGCGGCAGCGGGGGGGGCGGCAGGGGGGGGGGGGCAGCGGCGGCGGCG GCAGCQVQLKQSGAELARPGASVKLSCKASGYNFKSYGISWVKORTGQGLEWIGEIY PGSGNTYYNEKLKGKATLTADKSSSTAYMELRSLTSEDSAVYFCARTYGYDSFPWFA YWGQGTLVTVSS. The SEQ ID is NO. 9 DIVMTQAAPSVPVTPGESVSISCRSSKSLLHSNGNTYLYWFLQRPGQSPQLLIYRVSNL ASGVPDRFSGSGSGTAFTLRISRVEAEDVGVYYCMQHLEYPFTFGGGTKLEIKGGCG GCGGCGGCAGCGGCGGCGGCGGCAGCGGCGGCGGCGGCAGCGGCGGCGGCG GCAGCQVHLQQSGTELVKPGASVKLSCEASGYTFTSYWMHWVKQRPGQGLEWIGNI NPSNGGTNYNEKFKSKATLTVDKSSSTAYMQLSSLTSEDSAVYYCARGGIYYGNYDY YAMDYWGQGTSVTVSS. The present example obtains the lentivirus comprising the above two monoclonal antibodies, respectively.

In this example, the nucleotide sequence of the MSLN binding domain is shown in the SEQ ID NO. 5. The human primary T cells were commercially available.

### Example 3: Efficiency Verification

In this example, the lentivirus constructed from the plasmid AQP3-C ScFV-LIR1-T2A-MSLN scFv-CD8a-CD28-CD3z obtained from Example 2 was used to construct a stable cell line Hela-AQP3-GFP co-expressing MSLN with AQP3. Additionally, the Hela-mCherry cell line was also constructed for control. The core sequences and the vector types of both are shown in Table 1.

TABLE 1

| Gene | Vector | Sequence |
|---|---|---|
| Name | Type | (5′-3′) |
| empty | LV8N(EF-1a/ | Originating from plasmids |
| mCherry | mCherry&Puro) | https://www.addgene.org/25444/ |
| human-AQP3 | LV5(EF-1a/ | NCBI NM_004925.5 |
| | GFP&Puro) | |

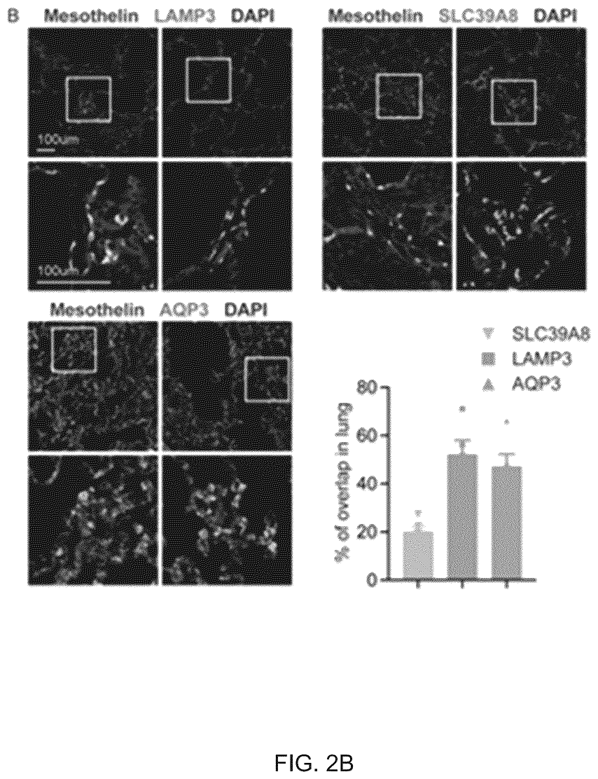

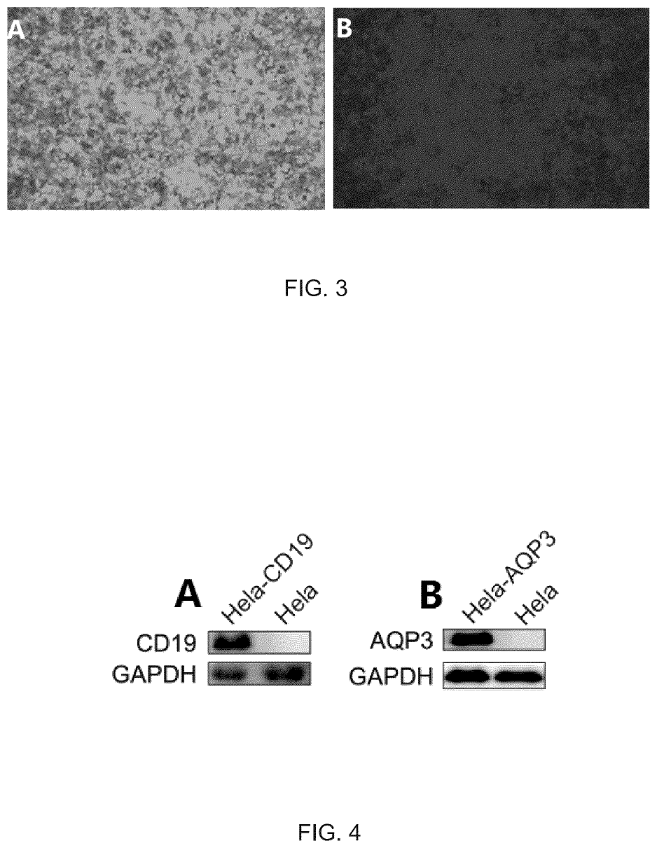

Specific steps for the construction of the Hela-AQP3-GFP cell line were as follows:
(1) After digesting cells in the logarithmic growth phase, 5*104 cells were added to each well in a 24-well plate using 0.5 mL complete medium and cultured overnight at 37° C. with a CO₂ concentration of 5%.
(2) 25 μL of virus stock solution and polybrene at a final concentration of 5 u g/mL were added to 400 μL of dilution solution (complete medium for target cells) to obtain the diluted virus solution. The cell culture medium was aspirated, and the diluted virus solution was added to the cells. At the same time, the control was established, i.e., target cells without virus solution was cultured in an incubator at 37° C. with a CO₂ concentration of 5% for 24 hours.
(3) After completing the culture, the virus solution was removed, 500 μL of complete culture medium was added and the culture was continued for 24 hours. Then puromycin-containing complete medium at a final concentration of 10 μg/mL was added and the culture was continued for 48 hours. The fluorescence results observed under the microscope are shown in FIG. 3. The cells were collected for Western Blot analysis, and the Western Blot image is shown in FIG. 4. FIG. 3 is a fluorescent image of cell lines according to some embodiments of the present disclosure. In the FIG. 3, A is a fluorescent image of the Hela-AQP3-GFP cell line and B is a fluorescent image of the Hela-mCherry cell line. FIG. 4 is a Western Blot image of cells collected after lentiviral transfection of a Hela cell line according to some embodiments of the present disclosure. In the FIG. 4, A is CD19 and B is the AQP3.

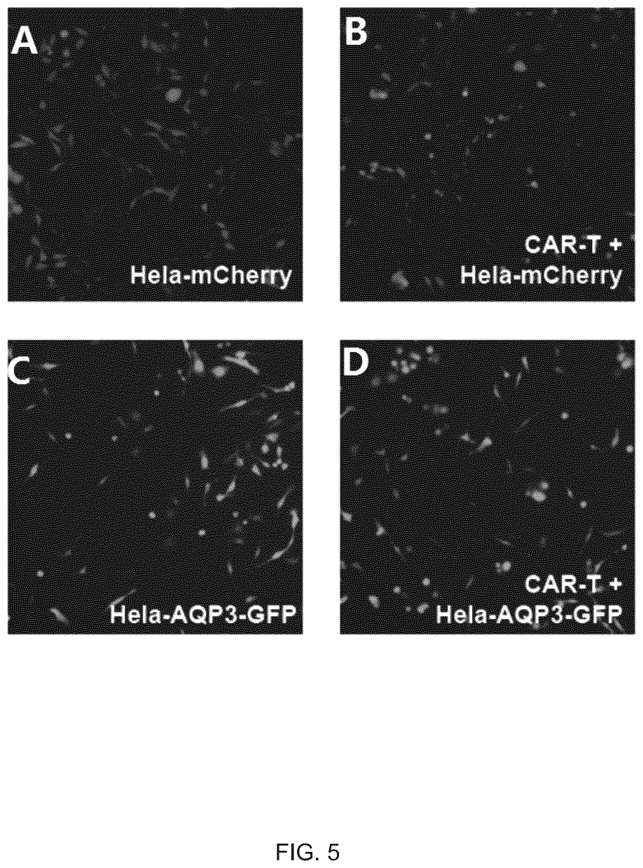

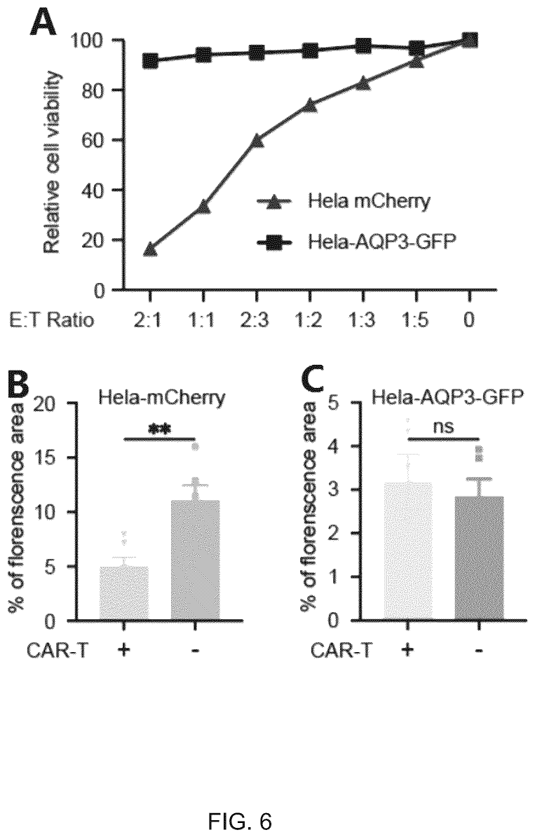

The human primary T cells infected with CAR lentiviruses were co-cultured with Hela cells transfected/untransfected with the AQP3 to detect the killing effect of the CAR-T cells on Hela cells and the protective effect of the CAR-T cells on the Hela-AQP3 cells. The obtained results are shown in FIGS. 5 and 6. FIG. 5 is a fluorescence comparison image of the killing effect of CAR-T cells on the Hela-AQP3-GFP cell line and the Hela-mCherry cell line according to some embodiments of the present disclosure. In the FIG. 5, A is the Hela-mCherry cell line without CAR-T cell infection, B is the Hela-mCherry cell line with CAR-T cell infection, C is the Hela-AQP3-GFP cell line with CAR-T cell infection, and D is the Hela-AQP3-GFP cell line with CAR-T cell infection. FIG. 6 is a comparison chart of the effects of the CAR-T cells on the Hela-AQP3-GFP cell line and the Hela-mCherry cell line according to some embodiments of the present disclosure. In the FIG. 6, A is a comparison chart of the relative cell viability, B is a fluorescence intensity of the Hela-mCherry cell line added with the CAR-T cells, and C is a fluorescence intensity of the Hela-AQP3-GFP cell line added with the CAR-T cells. According to B and C in FIG. 6, it can be seen that the fluorescence intensity of the Hela-mCherry cell line added with the CAR-T cells is decreased, which proves that the CAR-T cells kill target cells. However, there is no statistical difference between the fluorescence intensity of Hela-AQP3-GFP cell line added with the CAR-T cells and the Hela-AQP3-GFP cell line, which proves that the lung-exempt MSLN CAR-T cell could effectively protect the target cells from being killed.

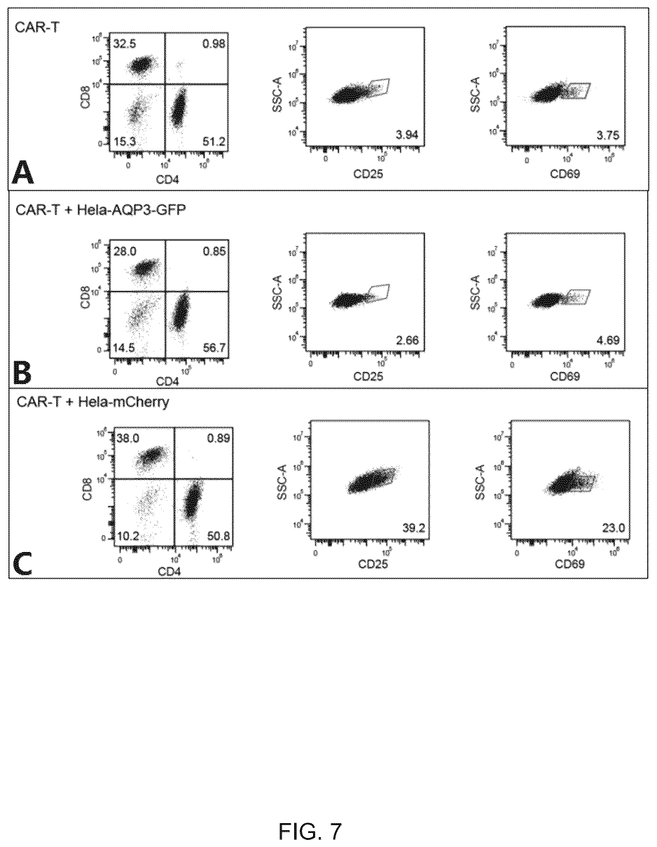

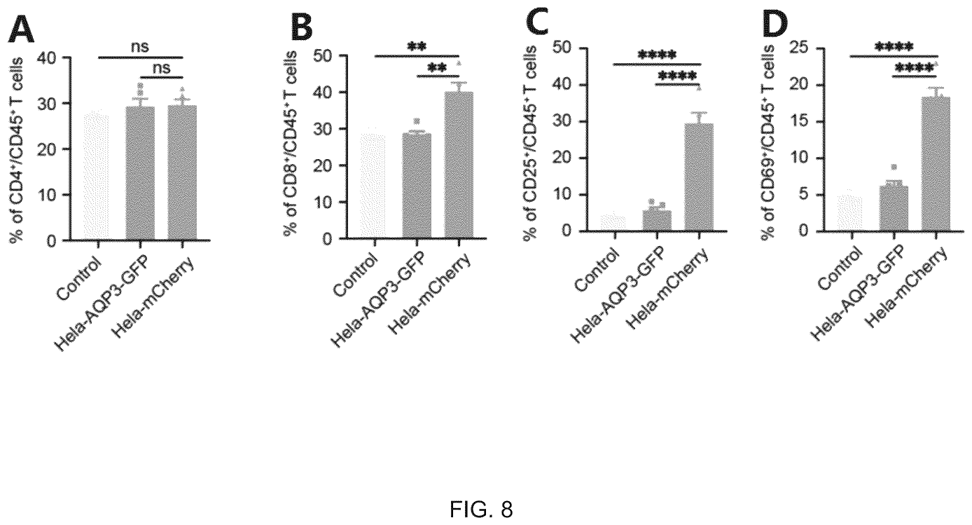

Supernatant T cells were collected for flow cytometry analysis, and the obtained results are shown in FIGS. 7 and 8. FIG. 7 is a schematic diagram illustrating the flow cytometry analysis results of T cells according to some embodiments of the present disclosure. In the FIG. 7, A is an analysis result diagram of the CAR-T cells, B is an analysis result diagram of the Hela-AQP3-GFP cell line with the CAR-T cell infection, and C is an analysis result diagram of the Hela-mCherry cell line with the CAR-T cell infection. FIG. 8 is a comparison graph of the different T cell contents in the experimental groups according to some embodiments of the present disclosure. In the FIG. 8, A-D illustrate activation and proliferation of CD4⁺ T cells, CD8⁺ T cells, CD25⁺ T cells, and CD69⁺ T cells after infection with Hela-AQP3 cells and Hela cells. Specifically, the proportions of these T-cell subsets in the CAR-T cell population may be determined, and the CAR-T cell population is defined as T cells expressing CD45+. According to FIG. 7 and FIG. 8, it can be seen that the T cells exhibit significant proliferation response after activation, which indicates that CAR-T cells are able to efficiently kill the Hela cells and does not exert killing efficacy on Hela-AQP3 cells.

As can be seen from the above embodiments, the lung-exempt MSLN CAR-T cell provided by the embodiments of the present disclosure can effectively kill cancer cells while significantly avoiding lung toxicity. For cancers that target MSLN other than ovarian cancer, the mechanism of action of the lung-exempt MSLN CAR-T cell is similar. Thus, for other cancers that target MSLN with concomitant low or non-expression of AQP3, the lung-exempt MSLN CAR-T cell has similar effects.

The basic concepts have been described above, apparently, in detail, as will be described above, and do not constitute limitations of the disclosure. Although there is no clear explanation here, those skilled in the art may make various modifications, improvements, and modifications of the present disclosure. This type of modification, improvement, and corrections are recommended in the present disclosure, so the modification, improvement, and the amendment remain in the spirit and scope of the exemplary embodiment of the present disclosure.

At the same time, the present disclosure uses specific words to describe the embodiments of the present disclosure. As “one embodiment,” “an embodiment,” and/or “some embodiments” means a certain feature, structure, or characteristic of at least one embodiment of the present disclosure. Therefore, it is emphasized and should be appreciated that two or more references to “an embodiment” or “one embodiment” or “an alternative embodiment” in various parts of the present disclosure are not necessarily all referring to the same embodiment. Further, certain features, structures, or features of one or more embodiments of the present disclosure may be combined.

In addition, unless clearly stated in the claims, the order of processing elements and sequences, the use of numbers and letters, or the use of other names in the present disclosure are not used to limit the order of the procedures and methods of the present disclosure. Although the above disclosure discusses through various examples what is currently considered to be a variety of useful embodiments of the disclosure, it is to be understood that such detail is solely for that purpose, and that the appended claims are not limited to the disclosed embodiments, but, on the contrary, are intended to cover modifications and equivalent arrangements that are within the spirit and scope of the disclosed embodiments.

Similarly, it should be appreciated that in the foregoing description of embodiments of the present disclosure, various features are sometimes grouped together in a single embodiment, figure, or description thereof for the purpose of streamlining the disclosure aiding in the understanding of one or more of the various embodiments. However, this disclosure does not mean that the present disclosure object requires more features than the features mentioned in the claims. Rather, claimed subject matter may lie in less than all features of a single foregoing disclosed embodiment.

In some embodiments, the numbers expressing quantities of ingredients, properties, and so forth, used to describe and claim certain embodiments of the application are to be understood as being modified in some instances by the term “about,” “approximate,” or “substantially.” Unless otherwise stated, “about,” “approximate,” or “substantially” may indicate ±20% variation of the value it describes. Accordingly, in some embodiments, the numerical parameters used in the specification and claims are approximate values, and the approximation may change according to the characteristics required by the individual embodiments. In some embodiments, the numerical parameter should consider the prescribed effective digits and adopt a general digit retention method. Although in some embodiments, the numerical fields and parameters used to confirm the breadth of its range are approximate values, in specific embodiments, such numerical values are set as accurately as possible within the feasible range.

With respect to each patent, patent application, patent application disclosure, and other material cited in the present disclosure, such as articles, books, manuals, publications, documents, etc., the entire contents thereof are hereby incorporated by reference into the present disclosure. Application history documents that are inconsistent with the contents of the present disclosure or that create conflicts are excluded, as are documents (currently or hereafter appended to the present disclosure) that limit the broadest scope of the claims of the present disclosure. It should be noted that in the event of any inconsistency or conflict between the descriptions, definitions, and/or use of terms in the materials appended to the present disclosure and those described in the present disclosure, the descriptions, definitions, and/or use of terms in the present disclosure shall prevail.

At last, it should be understood that the embodiments described in the present disclosure are merely illustrative of the principles of the embodiments of the present disclosure. Other modifications that may be employed may be within the scope of the present disclosure. Thus, by way of example, but not of limitation, alternative configurations of the embodiments of the present disclosure may be utilized in accordance with the teachings herein. Accordingly, embodiments of the present disclosure are not limited to that precisely as shown and described.

What is claimed is:

1. A recombinant vector, comprising a first nucleotide sequence, a second nucleotide sequence, and a third nucleotide sequence, wherein the first nucleotide sequence encodes a mesothelin (MSLN)-binding domain, the second nucleotide sequence encodes a leukocyte immunoglobulin-like receptor (LIR-1), and the third nucleotide sequence specifically binds to a lung-highly-expressed molecule.

2. The recombinant vector of claim 1, wherein the lung-highly-expressed molecule is AQP3.

3. The recombinant vector of claim 1, wherein the second nucleotide sequence is as shown in SEQ ID NO. 1.

4. The recombinant vector of claim 1, wherein the third nucleotide sequence is as shown in SEQ ID NO. 2 or SEQ ID NO. 3.

5. The recombinant vector of claim 1, wherein the first nucleotide sequence is as shown in SEQ ID NO. 5.

6. The recombinant vector of claim 1, wherein a 5′ end of the second nucleotide sequence is sequentially linked to a HINGE sequence and the third nucleotide sequence.

7. The recombinant vector of claim 6, wherein the HINGE sequence is as shown in SEQ ID NO. 7.

8. The recombinant vector of claim 1, wherein a 3′ end of the second nucleotide sequence is sequentially linked to a T2A linker sequence, the first nucleotide sequence, and an activating sequence.

9. The recombinant vector of claim 8, wherein the T2A linker sequence is as shown in SEQ ID NO. 4.

10. The recombinant vector of claim 8, wherein the activating sequence is as shown in SEQ ID NO. 6.

11. A lentivirus, wherein the lentivirus is constructed using the recombinant vector of claim 1.

12. A lung-exempt MSLN CAR-T cell, wherein the lung-exempt MSLN CAR-T cell is constructed using the recombinant vector of claim 1.

13. A lung-exempt MSLN CAR-T cell, wherein the lung-exempt MSLN CAR-T cell is constructed using the lentivirus of claim 11.

14. An anti-tumor agent, comprising the lung-exempt MSLN CAR-T cell of claim 12.

15. The agent of claim 14, wherein the agent is selected from at least one of a drug, a pharmaceutical raw material, a vaccine, a targeted inhibitor, a gene therapy vector, and a biological material, and the agent further comprises a pharmaceutically acceptable excipient.

16. An anti-tumor agent, comprising the lung-exempt MSLN CAR-T cell of claim 13.

17. The agent of claim 16, wherein the agent is selected from at least one of a drug, a pharmaceutical raw material, a vaccine, a targeted inhibitor, a gene therapy vector, and a biological material, and the agent further comprises a pharmaceutically acceptable excipient.
